(19) United States
(12) Patent Application Publication
(10) Pub. No.: US 2024/0034987 A1
(43) Pub. Date: Feb. 1, 2024

(54) SUPPLEMENTED SERUM-FREE MEDIA FOR CULTURED MEAT PRODUCTION

(71) Applicant: Trustees of Tufts College, Medford, MA (US)

(72) Inventors: David L. Kaplan, Concord, MA (US); Andrew Stout, Cambridge, MA (US); John Yuen, Somerville, MA (US); Addison Mirliani, Canton, MA (US)

(21) Appl. No.: 18/256,694

(22) PCT Filed: Dec. 9, 2021
(86) PCT No.: PCT/US21/62668

§ 371 Date: Jun. 9, 2023

Related U.S. Application Data
(60) Provisional application No. 63/123,346, filed on Dec. 9, 2020.

(51) Int. Cl. C12N 5/00; C12N 5/077

(52) U.S. Cl. CPC C12N 5/0043; C12N 5/0037; C12N 5/0659; C12N 2500/34; C12N 2500/38; C12N 2500/92; C12N 2500/25; C12N 2501/15; C12N 2501/115; C12N 2501/2306; C12N 2501/999; C12N 2501/998

(57) ABSTRACT

A serum-free and animal-component-free culture media for expansion of muscle satellite cells is disclosed. The disclosed media can be for use in cultured food applications. The disclosed media can include a baseline serum-free and animal component-free culture media and a recombinant version of albumin. The baseline media, in use, provides a baseline growth capability for expansion of the muscle satellite cells for use in cultured food applications. The disclosed media, in use, provides an improved growth capability for expansion of the muscle satellite cells. The improved growth capability is at least 50% greater than the baseline growth capability. Methods of making and using the disclosed media are also disclosed.

### CROSS-REFERENCE TO RELATED APPLICATIONS

This application is related to, claims priority to, and incorporates by reference herein for all purposes U.S. Provisional Patent Application No. 63/123,346, filed Dec. 9, 2020.

### STATEMENT REGARDING FEDERALLY FUNDED RESEARCH

Not applicable.

### BACKGROUND

Cell-cultured meat is an emerging technology which offers both promising possibilities and significant scientific challenges. The promise of cultured meat lies in its potential to address environmental, ethical, and human health issues that plague intensive animal agriculture. For instance, limited life-cycle analyses suggest that cultured meat could require >90% less land and >75% less water than conventional beef, while contributing >75% fewer greenhouse gas emissions, >95% less eutrophication, and >90% less particulate matter formation. At the same time, cultured meat could improve animal welfare, food-system resilience, and human health outcomes. The challenges that face the successful technological transition of cultured meat to the marketplace stem from the need for production systems that are low-cost, scalable, food-safe, and free of animal-derived inputs. Here, cell culture media is a particularly problematic hurdle for several reasons. First, media comprises the majority (>99%) of the cost of current production systems. Second, the culture of meat-relevant cells, such as bovine satellite cells (BSCs), has traditionally relied on fetal bovine serum (FBS), a notoriously expensive, unsustainable, and inconsistent component, which is inherently antithetical to the aims of cultured meat. Finally, when serum-free media for satellite cells have been explored, they are either complex, ineffective compared to serum-containing media, reliant on proprietary or animal-derived additives, or contain components (e.g., synthetic steroids) that could raise regulatory concerns. Further, no serum-free media has been validated for the sustained expansion of satellite cells over multiple passages. As such, despite the substantial body of work that has gone into the exploration of satellite cell culture systems, a food-safe and fully animal-derived component-free medium remains a crucial limitation for the field.

As a result, a need exists for new culture media.

### SUMMARY

In an aspect, the present disclosure provides a serum-free and animal-component-free culture media for expansion of muscle satellite cells for use in cultured food applications. The culture media includes a baseline serum-free and animal component-free culture media and a recombinant version of albumin. The baseline serum-free and animal-component-free culture media, in use, provides a baseline growth capability for expansion of the muscle satellite cells for use in cultured food applications. The culture media, in use, provides an improved growth capability for expansion of the muscle satellite cells for use in cultured food applications. The improved growth capability is at least 50% greater than the baseline growth capability.

In another aspect, the present disclosure provides a method of making a serum-free and animal-component free culture media. The method includes adding a recombinant version of albumin to a baseline serum-free and animal-component-free culture media. The adding produces the serum-free and animal-component-free culture media.

In another aspects, the present disclosure provides a method of making an engineered cell. The method includes expanding muscle satellite cells in the serum-free and animal-component-free culture media disclosed herein.

### BRIEF DESCRIPTION OF THE DRAWINGS

FIG. 1A is a plot of data showing short-term growth in BSC-GM mixed with B8, as discussed in Example 1. BSC proliferation over 3 & 4 days in mixtures of BSC-GM (20% FBS) and B8 media. At the four-day timepoint, mixtures of up to 62.5% B8 significantly improved growth compared to BSC-GM alone, and mixtures of up to 87.5% B8 did not significantly reduce growth (p=0.27). B8 alone showed a significant reduction in growth over four days compared with BSC-GM alone, and showed stagnating growth after three days. n=6 distinct samples; statistical significance was calculated by one-way ANOVA on day 4 data comparing all samples with BSC-GM controls, and is indicated by asterisks, in which p<0.05 (*), p<0.01 (**), p<0.001 (***), and p<0.0001 (****).

FIG. 1B shows brightfield images discussed in Example 1. Brightfield images of BSCs grown for three days in BSC-GM or B8 media. Images show that cell morphology was consistent in serum-containing or serum-free conditions. Cell confluency in the images is qualitatively consistent with growth analysis shown in FIG. 1A. Scale bars 200 μm.

FIG. 2A is a plot of data showing short-term growth in BSC-GM mixed with supplemented B8, as discussed in Example 1. BSC proliferation over 4 days B8 supplemented with Interleukin 6 (IL-6), curcumin, recombinant albumin (rAlbumin), linoleic acid, oleic acid, or mixtures of linoleic and oleic acid. Growth was analyzed on day 4 via dsDNA quantification, and values are given relative to B8. n=6 distinct samples; statistical significance was calculated by one-way ANOVA with multiple comparisons between supplemented samples and B8 controls, and is indicated by asterisks, in which p<0.05 (*), p<0.01 (**), p<0.001 (***), and p<0.0001 (****). While no significant difference was found for oleic acid on day 4 when tested alone (bottom middle panel), there was significant difference on day 3, and so it was included as part of the fatty acid mixture analysis (bottom right panel).

FIG. 2B is a plot of data showing short-term growth in BSC-GM mixed with supplemented B8, as discussed in Example 1. BSC proliferation over 4 days in B8 supplemented with combinations of factors, in which: B8=B8; I=IL-6 (0.01 ng/mL); A=rAlbumin (800 μg/mL); C=Curcumin (1 ng/mL); F=linoleic acid (400 ng/mL) and Oleic acid (400 ng/mL); and BSC-GM=serum-containing growth media. n=6 distinct samples; statistical significance was calculated by one-way ANOVA with multiple comparisons between all samples, and is indicated by asterisks, in which p<0.05 (*), p<0.01 (**), p<0.001 (***), and p<0.0001 (****). Statistically significant differences between B8 and other samples are shown, as is the lack of significance between rAlbumin supplemented B8 and BSC-GM (p>0.9990).

FIG. 2C shows a brightfield image discussed in Example 1. Brightfield imaging of BSCs at day 4 in B8 with rAlbumin (800 μg/mL) shows that cell morphology was maintained in B8 with albumin supplementation compared with images in FIG. 1B. Scale bar 200 μm.

FIG. 3A is a plot of data relating to passaging in Beefy-9, as described in Example 1. Growth analysis of BSCs passaged in B8/Beefy-9 media. Results showed that cells needed to be passaged in the absence of supplemental Albumin (“Delayed rAlbumin”), and that a coating (e.g., iMatrix-511 laminin) was required for adhesion and growth. Specifically, cells without any coating (“No coating”) were unable to grow, as were cells with iMatrix-511 coating that were passaged in the presence of albumin (“iMatrix-511; Passage w/rAlbumin”). In contrast, cells that were passaged onto iMatrix-511 coated flasks and allowed to adhere overnight before the addition of albumin (“Delayed rAlbumin”) showed exponential growth. n=9 image fields of view; statistical significance calculated by two-way ANOVA with multiple comparisons between conditions, and significant difference between “iMatrix-511; Delayed rAlbumin” and all other conditions are indicated by asterisks, in which p<0.05 (*), p<0.01 (**), p<0.001 (***), and p<0.0001 (****). A 95% confidence interval calculated via nonlinear regression (least squares regression; exponential (Malthusian) growth).

FIG. 3B is a schematic of a passaging system, as described in Example 1. Schematic of B8/Beefy-9 passaging system used. BSCs were plated in identical conditions (BSC-GM) on day 0 to ensure that an equal number of cells adhered to the plate initially. On day 1, cells were rinsed with PBS and media was changed to Beefy-9. At 70% confluency (day 3), cells were passaged using TrypLE and plated in B8 (no albumin) along with adhesive peptides. One day after passaging, media was changed to Beefy-9, and cells were proliferated and analyzed for adhesion, growth, and myogenicity.

FIG. 3C is a plot of data relating to passaging in Beefy-9, as described in Example 1. PrestoBlue adhesion and growth analysis of BSCs plated with various animal-free coatings. Truncated vitronectin (Vtn-N) at 1.5 μg/cm² showed superior cell attachment and growth compared to iMatrix-511 laminin (Lmn) or Poly-D-Lysine (PDL). n=3 distinct samples and 2 technical replicates; statistical significance was calculated by one-way ANOVA performed separately for day 1 or day 4 with multiple comparisons between Vtn-N 1.5 μg/cm² and all other samples. and is indicated by asterisks (day 1) or hashes (day 4), in which p<0.05 (*, #), p<0.01 (**, ##), p<0.001 (***, ###), and p<0.0001 (****, ####).

FIG. 4A shows plots of data illustrating short-term growth in B8 and Beefy-9 with reduced FGF-2, as discussed in Example 1. BSC proliferation over 4 days in B8 or Beefy-9 with various concentrations of FGF-2. FGF-2 could be reduced to 5 ng/mL or 1.25 ng/mL in B8 or Beefy-9, respectively, without significantly impacting cell growth over four days. n=6 distinct samples; statistical significance was calculated by one-way ANOVA with multiple comparisons between various FGF-2 concentrations and 40 ng/mL control conditions. Lack of significance between samples is indicated by ‘ns’ across all samples that hold no significant difference from 40 ng/mL).

FIG. 4B are brightfield images of short-term growth in Beefy-9 with no FGF-2 and reduced FGF-2, as discussed in Example 1. Brightfield images of BSCs grown for three days in Beefy-9 media with 0 or 5 ng/mL FGF-2. Images show that the complete removal of FGF-2 from Beefy-9 significantly affects cell morphology, whereas a reduction to 5 ng/mL did not affect morphology as compared with images in FIGS. 1B and 2C. Scale bars 200 μm.

FIG. 5A is data relating to long-term culture, as discussed in Example 1. Cell doublings over multiple passages of BSCs cultured in BSC-GM, B8, Beefy-9, high FGF (40 ng/mL FGF-2), or Beefy-9, low FGF (5 ng/mL FGF-2). Results show that the addition of albumin significantly improved cell growth in B8, although not to the degree of serum, over four weeks. Reducing Beefy-9 FGF-2 to 5 ng/mL decreased cell doublings compared to 40 ng/mL, although this difference was less substantial (17.2 doublings at 28 days for low FGF-2 vs 18.2 doublings for high FGF-2). n=6 (2 counts for 3 biological replicates), and error bars are given as ±standard deviation (though in some instances are smaller than the sample icons).

FIG. 5B is data relating to long-term culture, as discussed in Example 1. Doubling times were calculated over long-term cell culture and compared between media types. An increase in doubling time at higher passages was found, particularly for Beefy-9 with high or low FGF-2. Notably, however, doubling times remained <48 hours for the first five passages (˜13 doublings) in Beefy-9.

FIG. 6A is data relating to long-term culture with increased rAlbumin, as discussed in Example 1. Cell doublings over multiple passages of BSCs cultured in BSC-GM, Beefy-9 with rAlbumin concentrations of 0.8 mg/mL (data from FIG. 5), 1.6 mg/mL, 3.2 mg/mL, 6.4 mg/mL (with 40 or 5 ng/mL FGF-2), and HiDef Beefy-9 using supplier provided HiDef B8. Results showed that increased rAlbumin concentrations improved cell growth, with the highest numbers of cell doublings provided by concentrations of 6.4 mg/mL (20.2) and 3.2 mg/mL (19.8). n=6 (2 counts for 3 biological replicates), and error bars are given as±standard deviation (though in some instances are smaller than the sample icons).

FIG. 6B is data relating to long-term culture with increased rAlbumin, as discussed in Example 1. Final (28-day) cell counts for the media tested in (B), and with a starting cell population of 24,000. Results indicated significant increases in cell yield for 3.2 and 6.4 mg/mL rAlbumin compared with 0.8 mg/mL, as well as significant improvements when using engineered growth factors (in supplier provided HiDef B8). n=6 (2 counts for 3 biological replicates); statistical significance was calculated by one-way ANOVA with multiple comparisons between all conditions, and is indicated by asterisks, in which p<0.05 (*), p<0.01 (**), p<0.001 (***), and p<0.0001 (****).

FIG. 7 is a plot of data showing short-term growth in BSC-GM mixed with B8, as discussed in Example 1. BSC proliferation over 3 & 4 days in mixtures of BSC-GM (20% FBS) HiDef-B8 media. Results show that up to 50% HiDef-B8 significantly improves growth compared to BSC-GM, and that up to 87.5% does not significantly reduce growth compared to BSC-GM. n=6 distinct samples; statistical significance was calculated by one-way ANOVA on day 4 data comparing all samples with BSC-GM controls, and is indicated by asterisks, in which p<0.05 (*), p<0.01 (**), p<0.001 (***), and p<0.0001 (****).

FIG. 8 is a plot of data from a fusion index analysis of BSCs cultured over six passaged in various media, as discussed in Example 1. Fusion index of images (10× magnification) of BSCs cultured in various media and differentiated for 4 days (P2 and P4) or 6 days (P6). Fusion index given as the percent of imaged nuclei in the images that are superimposed with myosin heavy chain (MHC) staining after thresholding in ImageJ (threshold value=18 for all images). Results suggest improved differentiation in serum-free cultures; however, it is possible that reduced total cell number in serum-free conditions contribute to this conclusion, rather than solely differentiation extent. n=5-8 distinct images depending on whether any images were excluded due to imaging artifacts (e.g., bubbles) that clearly disrupted image analysis; statistical significance was calculated by two-way ANOVA with multiple comparisons between media types for each distinct passage tested, and is indicated by asterisks, in which p<0.05 (*), p<0.01 (**), p<0.001 (***), and p<0.0001 (****).

FIG. 9 is a plot showing short-term growth in Beefy-9 with additional rAlbumin. BSC proliferation over 3 & 4 days in increased concentrations of rAlbumin (over 800 ug/mL, as was formulated for the original Beefy-9 medium. Results show an drastic increase in growth with increasing rAlbumin concentration. Indeed, while 11.2 mg/mL was the maximum concentration tested, this was not the limit for improving short-term growth. n=6 distinct samples; statistical significance was calculated by one-way ANOVA on day 4 data comparing all samples with Beefy-9 (“800”), and is indicated by asterisks, in which p<0.05 (*), p<0.01 (**), p<0.001 (***), and p<0.0001 (****).

### DETAILED DESCRIPTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

In one aspect, the present disclosure provides a serum-free and animal-component-free culture media. The inventive culture media is based off the discovery that a recently-developed serum-free and animal-product-free culture media for induced pluripotent stem cells, which was not it its original form suitable for commercial-scale growth and expansion of muscle satellite cells, could be relatively easily modified by the addition of a recombinant form of albumin for growth and expansion of muscle satellite cells. The inventive culture media may be suitable for commercial production of cultured muscle cells.

The recombinant version of albumin can be present in an amount of at least 50 mg/L, at least 100 mg/L, at least 200 mg/L, at least 250 mg/L, at least 500 mg/L, at least 750 mg/L, at least 1 g/L, at least 2 g/L, at least 5 g/L, at least 10 g/L, at least 15 g/L, or at least 20 g/L and at most 60 g/L, at most 50 g/L, at most 40 g/L, at most 30 g/L, at most 25 g/L, at most 20 g/L, at most 15 g/L, at most 10 g/L, at most 5 g/L, at most 2 g/L, at most 1.5 g/L, or at most 1 g/L. In some cases, the recombinant version of albumin is present in an amount of about 800 mg/L. In some cases, the recombinant version of albumin is present in an amount of between 800 mg/L and 6400 mg/L.

The recombinant version of albumin can be a recombinant version of human albumin, bovine albumin, porcine albumin, chicken albumin, or another albumin that a skilled artisan would recognize as being likely to behave similarly to the disclosed albumins. In some cases, the albumin can be an albumin or similar protein from plant and non-animal species.

In some cases, the serum-free and animal-component-free culture media can further include a recombinant version of one or more of the following: interleukin 6; ethanolamine; curcumin; oleic acid; or linoleic acid.

The serum-free and animal-component-free culture media and the baseline culture media can include basal media in an amount by weight of at least 80% and at most 99.99%.

The baseline serum-free and animal-component-free culture media can contain a mixture of basal media (e.g., DMEM, DMEM/F12, etc.) containing sugars (e.g., glucose) at concentrations ranging from 0.01-10 g/L; amino acids (e.g., glutamine, lysine) at concentrations ranging from 0.001-5 g/L, vitamins (e.g., folate, niacin) at concentrations ranging from 0.001-1 g/L; minerals (e.g., NaCl) at concentrations ranging from 0-15 g/L; and trace elements (e.g., iron, selenium) at concentrations ranging from 0.0001-10 mg/L. The basal media can be supplemented with growth-stimulating or cell-signaling factors (e.g., insulin, fibroblast growth factor, transforming growth factor, etc.) at concentrations ranging from 0.01-100,000 ng/mL and by carrying proteins (e.g., transferrin) at concentrations ranging from 0.01-1 g/L.

The baseline serum-free and animal-component-free culture media has a baseline growth capability for expansion of muscle satellite cells for use in cultured food applications. However, this baseline growth capability is unsuitable for commercial growth of cultured food. The disclosed serum-free and animal-component-free culture media has an improved growth capacity that is at least 50% greater than the baseline growth capacity. In some cases, the improved growth capacity is at least 100%, at least 150%, at least 200%, at least 250%, or at least 300% greater than the baseline growth capacity.

The baseline and improved growth capacity can be expressed in a variety of ways. As one example, the baseline and improved growth capacity can be expressed as a short-term growth capacity, measured as proliferation over the course of a short period of time, such as 1, 2, 3, 4, 5, 6, or 7 days. As another example, the baseline and improved growth capacity can be expressed as a long-term growth capacity, measured as a number of cell doublings over multiple passages of muscle satellite cells. As a further example, the baseline and improve growth capacity can be expressed as a biomass increase over a given period of time. As yet another example, the baseline and improved growth capacity can be expressed as a percentage of actively doubling cells in culture at a given time (e.g., through cell-cycle analysis). The specific way in which the growth capacity is expressed is not intended to be limiting.

In certain aspects, the culture media can include FGF-2 at a lower concentration than would ordinarily be present in the baseline culture media. In the baseline culture media, the FGF-2 concentration is typically around 40 ng/mL. The inventors surprisingly discovered that, with the inventive culture media including a recombinant form of albumin, the concentration of FGF-2 in the culture media can be less than 20 ng/mL, less than 15 ng/mL, less than 10 ng/mL, less than 7.5 ng/mL, less than 5 ng/mL, or less than 2.5 ng/mL.

In certain aspects, the culture media can include transforming growth factor (TGFβ3) at a lower concentration than would ordinarily be present in the baseline culture media. In some cases, the culture media can include TGFβ3 at a concentration of less than 0.1 ng/mL, less than 0.01 ng/mL, less than 0.001 ng/mL. In some cases, the culture media can include no TGFβ3.

In certain cases, the culture media can include neuregulin (NRG1) at a lower concentration than would ordinarily be present in the baseline culture media. In some cases, the culture media can include NRG1 at a concentration of less than 0.1 ng/mL, less than 0.01 ng/mL, less than 0.001 ng/mL. In some cases, the culture media can include no NRG1.

In some cases, the culture media can include other components at a lower concentration than would ordinarily be present in the baseline culture media.

In another aspect, the present disclosure provides a method of using the culture media disclosed herein. In some cases, this is a method of making an engineered cell. In some cases, the culture media disclosed herein can be used for expansion of muscle cells for use as food ingredients (e.g., cultured meat/seafood, or as supplements to add to plant-based meat products). In some cases, the culture media disclosed herein can be used for expansion of muscle cells in a bioreactor, either on hollow fibers or microcarriers or in single-cell suspension or cell aggregate suspension (e.g., stirred-tank bioreactors, fluidized bed bioreactors, hollow-fiber bioreactors, rotating-wall bioreactors, wave bioreactors, packed-bed bioreactors, airlift bioreactors, etc.). In some cases, the culture media disclosed herein can be used for expansion of muscle cells for regenerative medicine applications (e.g., in the above-described bioreactors for the treatment of volumetric muscle loss). In certain cases, the culture media disclosed herein can be used as isolation media for generating primary muscle cell populations.

A method of making an engineered cell can include expanding muscle satellite cells in the serum-free and animal-component-free culture media disclosed herein. Prior to expanding the muscle cells, the method can further include either: i) coating the muscle satellite cells with a cell adhesive peptide; or ii) adhering the muscle satellite cells in the baseline serum-free and animal-component-free culture media and/or a different baseline serum-free and animal-component-free culture media that is lacking the recombinant version of albumin.

The cell adhesive peptide can be a recombinant version of a laminim (e.g., laminin 511) or fragments thereof, vitronectin or fragments thereof, poly-d-lysine, poly-l-lysine, fibronectin or fragmetns thereof, Matrigel, or other cell adhesive peptides understood by those skilled in the art

The adhering of step ii) can be performed in B8 culture media or other media that a skilled artisan would recognize as suitable for such adhering. Examples of other suitable media include essential 8 media, TeSR-E8 media, basal media (e.g., DMEM, DMEM/F12, etc.), proprietary serum-free media, serum-containing culture media, and other media understood by a skilled artisan to be suitable for adhering.

The muscle satellite cells discussed herein with respect to the culture media and the methods can be from an animal source, including, without limitation, from bovine, avian (e.g., chicken, quail), porcine, seafood, or murine sources. The muscle satellite cells discussed herein with respect to the culture media and the methods can be derived from seafood such as fish (e.g., salmon, tuna, tilapia, perch, mackerel, cod, sardine, trout, etc.), shellfish (e.g., clams, mussels, and oysters); crustaceans (e.g., lobsters, shrimp, prawns, and crayfish), and echinoderms (e.g., sea urchins and sea cucumbers). The muscle satellite cells discussed herein with respect to the culture media and the methods can be bovine, galline, ovine, porcine, equine, murine, caprine, lapine, or piscine. In some cases, the muscle satellite cells are bovine, galline, porcine, or piscine.

In another aspect, the present disclosure provide a method of making a serum-free and animal-component-free culture media. The method includes adding a recombinant version of albumin to a baseline serum-free and animal-component free culture media, thereby producing the serum-free and animal-component-free culture media. In some cases, this can involve mixing all of the components of the serum-free and animal-component-free culture media. In some cases, certain portions can be pre-mixed before combining with other portions. A skilled artisan will recognize that the specific method of making the inventive culture media of the present disclosure is not intended to be limiting to the protection of the culture media or the method of using the culture media.

Before discussing the exemplified aspects of the present disclosure, Applicant emphasizes that the inventiveness of the present disclosure lies heavily with the fact that the inventive culture media has been validated. Without wishing to be bound by a particular theory, we remain at the dawn of cultured meat products and it remains very challenging to predict efficacy, particularly when it relates to formulations that traditionally involve serum or animal products. Prior to this invention, to the best of Applicant's knowledge no successful culture media had been developed for long-term propagation muscle satellite cells without the use of serum, other animal products, or non-food-safe components. Additionally, prior to this invention, to the best of Applicant's knowledge no method for the passaging and propagation of these cells in a serum-free manor had been developed, and indeed the requirement for albumin's temporary absence from the culture medium during cell passaging is nonobvious and represents significant methodological novelty. As a result, the number of possibilities remains nearly infinite while the guidance from successful examples is practically zero. Because of this, Applicant submits that the bar for what might be considered inventive in this space needs to properly consider these factors.

### EXAMPLES

### Example 1

Materials and Methods

Primary Bovine Satellite Cell Isolation and Maintenance

Primary bovine satellite cells (BSCs) were isolated with methods previously used in our group and based on previously described pre-plating satellite-cell isolation protocols. See, e.g., Stout, A. J., Mirliani, A. B., Soule-Albridge, E. L., Cohen, J. M. & Kaplan, D. L. Engineering carotenoid production in mammalian cells for nutritionally enhanced cell-cultured foods. Accept.—Metab. Eng. 62, 126-137 (2020) and Gharaibeh, B. et al. Isolation of a slowly adhering cell fraction containing stem cells from murine skeletal muscle by the preplate technique. Nat. Protoc. 2008 39 3, 1501-1509 (2008). Briefly, ˜0.5 g of muscle was excised from the semitendinosus of a 14-day-old Simmental calf at the Tufts Cummings School of Veterinary Medicine according to approved methods (IACUC protocol #G2018-36). Muscle tissue was minced into a paste and digested in 0.2% collagenase II (Worthington Biochemical #LS004176, Lakewood, NJ, USA; 275 U/mg) for 45 minutes with regular trituration. Digestion was halted with BSC growth media (BSC-GM) comprised of DMEM+Glutamax (ThermoFisher #10566024, Waltham, MA, USA) supplemented with 20% fetal bovine serum (FBS; ThermoFisher #26140079), 1 ng/mL human FGF-2 (ThermoFisher #68-8785-63), and 1% Primocin (Invivogen #ant-pm-1, San Diego, CA, USA), and cells were filtered and plated at a density of 100,000 cells/cm² onto uncoated tissue-culture flasks. After 24 hours of incubation at 37° C. with 5% C02, the plated suspensions (containing satellite cells) were transferred to flasks coated with 1 μg/cm² mouse laminin (Sigma #CC095, St. Louis, MO, USA), which were left untouched for three days before growth media was changed, and cells were cultured using standard practices on tissue-culture plastic coated with 0.25 ug/cm² iMatrix recombinant laminin-511 (Iwai North America #N892021, San Carlos, CA, USA). After two weeks of culture, Primocin in growth media was replaced with 1% antibiotic-antimycotic (ThermoFisher #1540062). For regular cell maintenance, cells were cultured at 37° C. in 5% C02 to a maximum of 70% confluence, counted using an NC-200 automated cell counter (Chemometec, Allerod, Denmark), and either passaged using 0.25% trypsin-EDTA (ThermoFisher #25200056) or frozen in FBS with 10% Dimethyl sulfoxide (DMSO, Sigma #D2650). For routine myogenic differentiation, cells were cultured to confluency as above and then incubated for one week without changing the medium.

Characterization of Isolated Cells

To characterize isolated cells, proliferative BSCs were stained for Paired-box 7 (Pax7), a marker of satellite cell identity. Cells were fixed with 4% paraformaldehyde (ThermoFisher #AAJ61899AK) for 30 minutes, washed in PBS, permeabilized for 15 minutes using 0.5% Triton-X (Sigma #T8787) in PBS, blocked for 45 minutes using 5% goat serum (ThermoFisher #16210064) in PBS with 0.05% sodium azide (Sigma #S2002), and washed with PBS containing 0.1% Tween-20 (Sigma #P1379). Primary Pax7 antibodies (ThermoFisher #PA5-68506) were diluted 1:100 in blocking solution containing 1:100 Phalloidin 594 (ThermoFisher #A12381), added to cells, and incubated overnight at 4° C. Cells were then washed with PBS+Tween-20, incubated with secondary antibodies for Pax7 (ThermoFisher #A-11008, 1:500) for 1 hour at room temperature, washed with PBS+tween-20, and mounted with Fluoroshield mounting medium with DAPI (Abcam #ab104139, Cambridge, UK) before imaging. Imaging was performed via fluorescence microscopy (KEYENCE, BZ-X700, Osaka, Japan). Batch colocalization analysis was performed on multiple images using the BZ-X800 image cytometry software to enumerate the nuclei which were positive for Pax7, thereby giving a quantitative measure of satellite cell purity in the isolated cell population.

To validate myogenicity of isolated BSCs, cells were differentiated for 7 days as described. Cells were then fixed, stained, and imaged as previously described, using primary antibodies for myosin heavy chain (Developmental studies hybridoma bank #MF-20, Iowa City, IA, USA), phalloidin 594 (1:100), appropriate secondary antibodies (ThermoFisher #A-11001, 1:1000), and Fluoroshield mounting medium with DAPI.

Short-Term Growth Analysis

Homemade B8 medium was prepared using store-bought components and a previously described formulation and method of preparation. See, Kuo, H. H. et al. Negligible-Cost and Weekend-Free Chemically Defined Human iPSC Culture. Stem Cell Reports 14, 256-270. Table 1 includes the formulation. Additionally, HiDef-B8 medium aliquots were generously provided by Defined Bioscience (Defined Bioscience #LSS-201, San Diego, CA, USA) and added DMEM/F12 with 1% antibiotic/antimycotic. Short term BSC growth (3 and 4 days) was analyzed for mixtures of serum-containing and serum-free media, as well as for pure B8 media with reduced growth factor concentrations and/or with the addition of various media supplements (Table 2). Briefly, BSCs were thawed (passage number <2) and plated in BSC-GM on 96-well tissue-culture plastic plates for each timepoint at a density of 2,500 cells/cm² with 0.25 ug/cm² iMatrix recombinant laminin-511. After 24 hours, BSC-GM was removed, cells were washed 1× with DPBS, and new media (e.g., B8+/−supplementation) was added. A list of supplements and concentrations can be found in Table 2. Media was changed on day 3, and on days 3 and 4 cells were imaged, media was aspirated from appropriate plates, and plates were frozen at −80° C. Once all timepoints were frozen, cell number was analyzed using a FluoReporter dsDNA quantitation kit (ThermoFisher #F2962) according to the recommended protocol and with fluorescence readings performed on a Synergy H1 microplate reader (BioTek Instruments, Winooski, VT, USA) using excitation and emission filters centered at 360 and 490 nm, respectively. Cell number at 3 and 4 days was analyzed relative to pure B8 or HiDef media.

TABLE 1

| Component | Concentration | | Supplier | Catalog # |
|---|---|---|---|---|
| DMEM/F12 basal media | N/A | | ThermoFisher | 11320033 |
| 2-Phospho-L-ascorbic acid trisodium salt | 200 | μg/mL | Sigma | 49752-10G |
| Insulin (human, recombinant) | 20 | μg/mL | Sigma | 91077C-250MG |
| Transferrin (human, recombinant) | 20 | μg/mL | InVitria | 777TRF029 |
| Sodium selenite | 20 | ng/mL | Sigma | S5261-10G |
| Fibroblast growth factor (FGF-2) | 40 | ng/mL | PeproTech | 100-18B |
| Neuregulin (NRG1) | 0.1 | ng/mL | PeproTech | 100-03 |
| Transforming growth factor (TGFβ3) | 0.1 | ng/mL | R&D Systems | 8420-B3-005/CF |
| UltraPure Water | 5.8% | (v/v) | ThermoFisher | 10977015 |
| Antibiotic/Antimycotic | 1% | (v/v) | ThermoFisher | 1540062 |

Functional species in media components are bolded. To prepare media, two aliquots were first prepared. For aliquot ‘A,’ 400 mg/mL 2-Phospho-L-ascorbic acid trisodium salt was slowly prepared in water, sterilized, and aliquoted in 250 μL portions. For aliquot ‘B,’ 40 mg/mL insulin was added to water, and 1 N HCl was added until insulin had dissolved. Slowly, 1 N NaOH was added to bring the pH up to ˜6. Next, 40 mg/mL transferrin, 40 μg/mL sodium selenite, 80 μg/mL FGF-2, 0.2 μg/mL NRG1, and 0.2 μg/mL TGFβ3 were added and the solution was sterilized and aliquoted in 250 μL portions. To prepare media, DMEM/F12 (500 mL) was sterilized with 5.3 mL of 100× Antibiotic/Antimycotic and 31 mL of water through a sterile filter, and aliquots A and B were added after sterilization. Following protocol previously described for B8 media, media used in this study was used within one month of preparing from frozen aliquots, and media was warmed to room temperature before feeding cells.

TABLE 2

| Component | Supplier | Catalogue # | Range (ng/mL) |
|---|---|---|---|
| Hydrocortisone | Sigma | H0888-1G | 3.125-100 |
| Estradiol | Sigma | E8875-1G | 1-5,000 |
| Progesterone | Sigma | P8783-1G | 0.3125-10 |
| Dexamethasone | Sigma | D4902-100MG | 3.125-400 |
| BMS 564929 | Tocris Bioscience | 5274 | 0.1-500 |
| Bovine growth hormone | MP Biomedical | 02160074.1 | 7.8125-250 |
| Curcumin | Sigma | C7727-500MG | 1-5,000 |
| Spermidine | Sigma | S2626-1G | 100-500,000 |
| Ethanolamine | Acros Organics | 149582500 | 10-50,000 |
| Linoleic acid | Sigma | L5900-10MG | 25-3,200 |
| Oleic acid | Sigma | O1257-10MG | 25-3,200 |
| Stat3 inhibitor | Sigma | 573096-1MG | 100-500,000 |
| Interleukin-6 | BioRad | PBP021 | 0.01-50 |
| Leukemia inhibitory factor | Peprotech | 300-05 | 0.3125-40 |
| Hepatocyte growth factor | Novus Biologicals | NBP2517300.1MG | 0.3125-40 |
| Platelet derived growth factor BB | ThermoFisher | PHG0045 | 0.15625-20 |
| Pigment-epithelium derived factor | R&D Systems | 1177SF025 | 0.390625-50 |
| Insulin-like growth factor | ThermoFisher | PHG0078 | 0.1-50 |
| Cardiotrophin 1 | Novus Biologicals | NBP199745 | 0.15625-20 |
| Recombinant human albumin | Sigma | A9731-1G | 50,000-60,000,000 |

Passaging in Beefy-9 Media

To test various passaging conditions using B8+rAlbumin (Beefy-9), BSCs were plated in BSC-GM onto T-75 culture flasks at a density of 2,500 cells/cm² with 0.25 ug/cm² iMatrix recombinant laminin-511. After 24 hours, BSC-GM was removed, cells were washed 1× with DPBS, and Beefy-9 media was added. Cells were cultured to 70% confluency, harvested with TrypLE Express (ThermoFisher #12604021), centrifuged at 300 g, and resuspended in B8 or Beefy-9 media with or without iMatrix laminin-511. Cells were seeded at 5,000 cells/cm² (0.25 ug/cm² iMatrix laminin) onto a 12-well plate, and growth was analyzed with a live cell monitoring system (Olympus Provi CM20, Tokyo, Japan). After 24 hours, media was aspirated, and all cells were fed with Beefy-9 media. Cell growth was compared over seven days in order to determine the effects of seeding+/−rAlbumin and +/−iMatrix laminin.

To test the effect of different coatings, 48-well plates were prepared with or without pre-coating with poly-D-lysine (Sigma #P1024-10MG) according to the manufacturer's instructions. Cells were then cultured and harvested as above, centrifuged at 300 g, and resuspended in B8 media with varying concentrations if iMatrix laminin and/or truncated recombinant human vitronectin (ThermoFisher #A14700). Cells were seeded at 5,000 cells/cm². After 24 hours, media was aspirated, and cells were rinsed 1× with DPBS. Cells were then fed with Beefy-9 media with 10% PrestoBlue reagent (ThermoFisher #A13262) and incubated at 37° C. After 2.5 hours, PrestoBlue media was moved to a 96-well plate and read with a Synergy H1 microplate reader using excitation and emission filters centered at 560 and 590 nm, respectively. Cell culture media was then replenished with Beefy-9 and PrestoBlue analysis was repeated on Day 4.

Long-Term Growth Analysis

For generating long-term growth curves, BSCs were thawed and plated (P1) onto 6-well culture plates (triplicate wells) in BSC-GM with 0.25 ug/cm² iMatrix laminin-511. After allowing cells to adhere overnight, media was removed, cells were washed 1× with DPBS, and either BSC-GM, B8, Beefy-9 (40 ng/mL FGF-2) or Beefy-9 (5 ng/mL FGF-2) were added to cells. Upon reaching ˜70% confluency, cells were rinsed with DPBS, harvested with TrypLE Express, and counted using an NC-200 automated cell counter (duplicate counts for each well). Cells were then pelleted at 300 g, resuspended in BSC-GM or B8 media, re-counted, and seeded onto new 6-well plates at 2,500 cells/cm² with 0.25 ug/cm² iMatrix laminin-511 (cells in BSC-GM) or 1.5 ug/cm² recombinant vitronectin (cells in B8). After allowing cells to adhere overnight, media was replaced with appropriate media (BSC-GM, B8, Beefy-9 (40 ng/mL FGF-2) or Beefy-9 (5 ng/mL FGF-2). This process was repeated over 28 days and seven passages. Throughout culture, cells were fed every two days. When seeding cells for passages 2, 4, 6, and 7, additional wells were seeded for staining for myosin heavy chain (P2, P4 and P6) or lipid accumulation (P7).

Serum-Free Differentiation

Throughout long-term culture, a population of cells at passages 2, 4, and 6 were cultured to confluency in serum-containing or serum-free conditions, and media was changed to a previously described serum-free differentiation media consisting of Neurobasal (Invitrogen #21103049, Carlsbad, CA, USA) and L15 (Invitrogen #11415064) basal media (1:1) supplemented with 1% antibiotic/antimycotic, 10 ng/mL insulin-like growth factor 1 (IGF-1; Shenandoah Biotechnology #100-34AF-100UG, Warminster, PA, USA) and 100 ng/mL epidermal growth factor (EGF; Shenandoah Biotechnology #100-26-500UG). Cells were differentiated for 4-6 days (changing media every two days).

Characterization of Cultured & Differentiated Cells

To verify myogenicity of cells differentiated in serum-free media, cells from above were fixed, stained, and imaged as previously described, using primary antibodies for MHC (Developmental studies hybridoma bank #MF-20), phalloidin 594 (1:100), appropriate secondary antibodies (ThermoFisher #A-11001, 1:1000), and Fluoroshield mounting medium with DAPI.

To analyze lipid accumulation in culture, media was replaced with PBS containing 2 μM BODIPY™ 493/503 (ThermoFisher #D3922). Cells were incubated for 20 minutes at 37° C., washed 2× with PBS, and fixed for 30 minutes with 4% paraformaldehyde. Cells were then washed 3× with PBS, permeabilized and blocked as before, and incubated at room temperature for one hour in blocking solution containing phalloidin 594 (1:100) and 1 μg/mL DAPI (ThermoFisher #62248). Stained cells were rinsed 3× with PBS and imaged via fluorescence microscopy as before.

Statistical Analysis

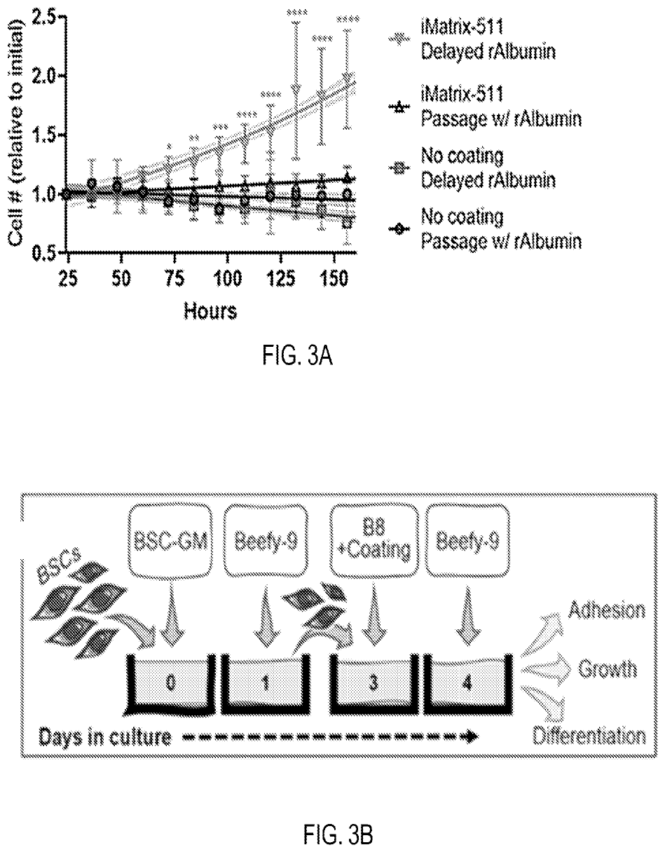

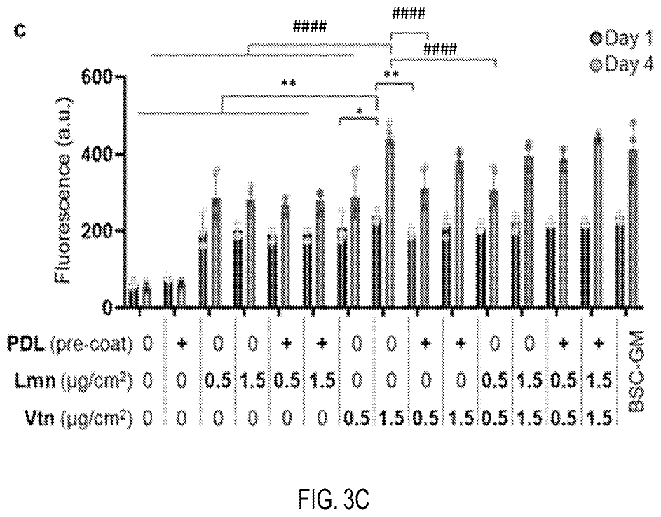

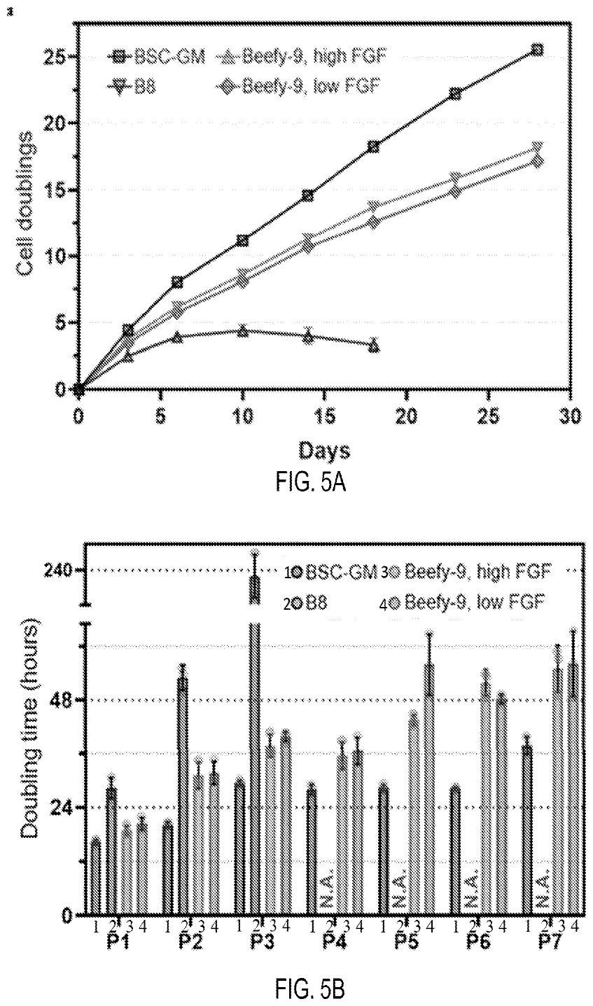

Statistical analysis was performed with GraphPad Prism 9.0 software (San Diego, CA, USA). Short-term cell growth analyses were performed via one-way or two-way ANOVA, as appropriate, with multiple comparisons performed with the Tukey's HSD post-hoc test. Regression analysis (FIG. 3A) was performed via nonlinear regression (least squares; exponential (Malthusian) growth) and is shown alongside 95% confidence interval. Doubling time (FIG. 5B) was determined through nonlinear regression (least squares; exponential (Malthusian) growth) for each biological replicate (well) of each passage, using technical replicates (duplicate counts) in generating nonlinear regressions. P values <0.05 were treated as significant. Unless otherwise stated, errors are given as ±standard deviation.

Results

B8 media can lower serum requirements for BCS's during short-term growth.

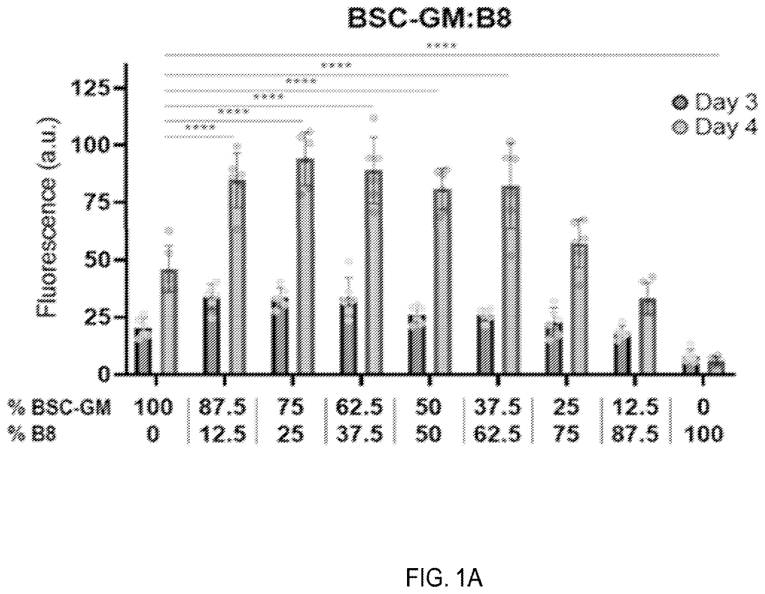

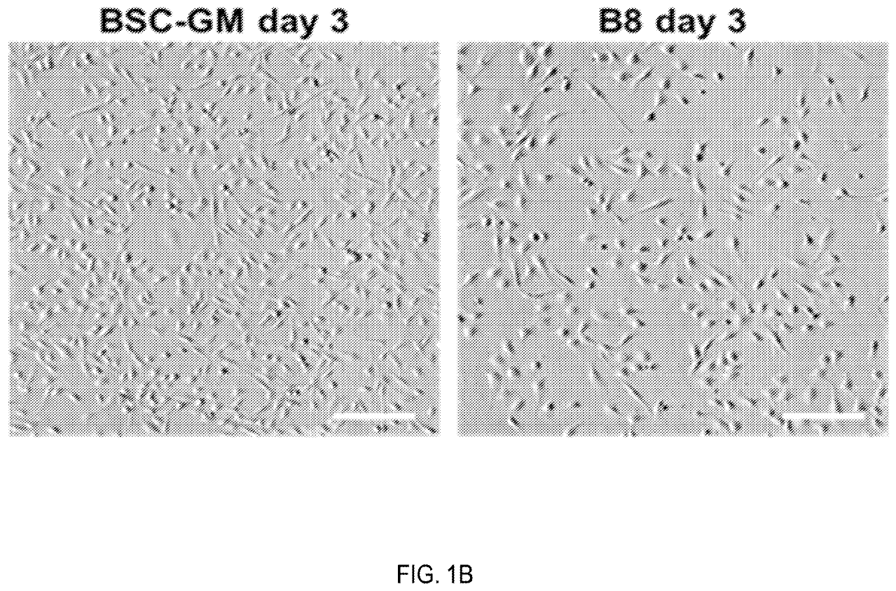

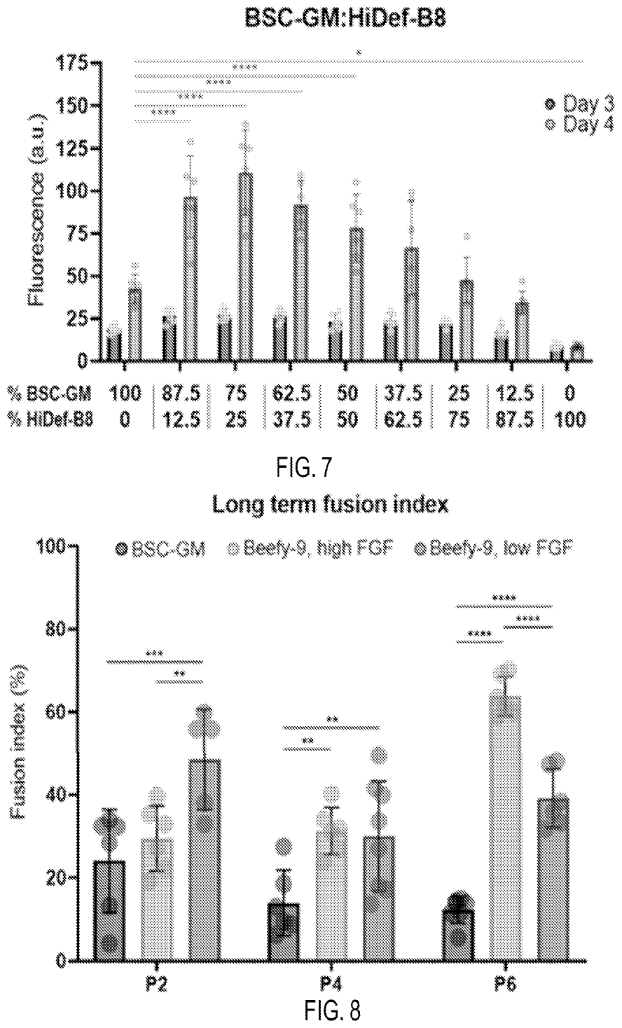

Bovine satellite cells were used throughout experiments. First, staining for Pax7 and Myosin Heavy Chain (MHC) were performed before and after differentiation of isolated cells in order to verify the initial and terminal states of these stem cells. See, Relaix, F., Rocancourt, D., Mansouri, A. & Buckingham, M. A Pax3/Pax7-dependent population of skeletal muscle progenitor cells. Nat. 2005 4357044 435, 948-953 (2005) and Jankowski, R. J., Deasy, B. M. & Huard, J. Muscle-derived stem cells. Gene Ther. 9, 642-647 (2002). Quantitative image cytometry revealed that 96.3% of isolated cells were positive for Pax7 (images corresponding to this analysis can be provided to a patent office, if necessary, and can be located by any reader after the publication of this patent application by searching for a journal article by the inventors and other co-authors describing these experiments), indicating a highly pure population of satellite cells. After cell identity was verified, B8's capacity to replace serum containing media was analyzed via short term BSC growth assays (3 and 4 days) in mixtures of BSC-GM combined with homemade B8 or supplier provided HiDef-B8 (FIG. 1 & FIG. 7). These timepoints were selected because cells in fast-growing conditions reached confluency by day 5 (data not shown). The results showed that B8 media mixed with BSC-GM significantly improved growth compared to BSC-GM alone over four days (FIG. 1A), and that this benefit remained with as much as a 62.5% reduction in FBS (62.5% B8 media). Additionally, an 87.5% reduction in serum did not significantly reduce cell growth over four days. On the other hand, while B8 media alone encouraged cell growth over three days (FIGS. 1A & B), there was no change between days 3 and 4, indicating that growth did not continue into day 4. These results indicated that B8 was capable of reducing serum requirements in BSCs, but could not completely eliminate these requirements.

B8 media supplementation improves cell proliferation.

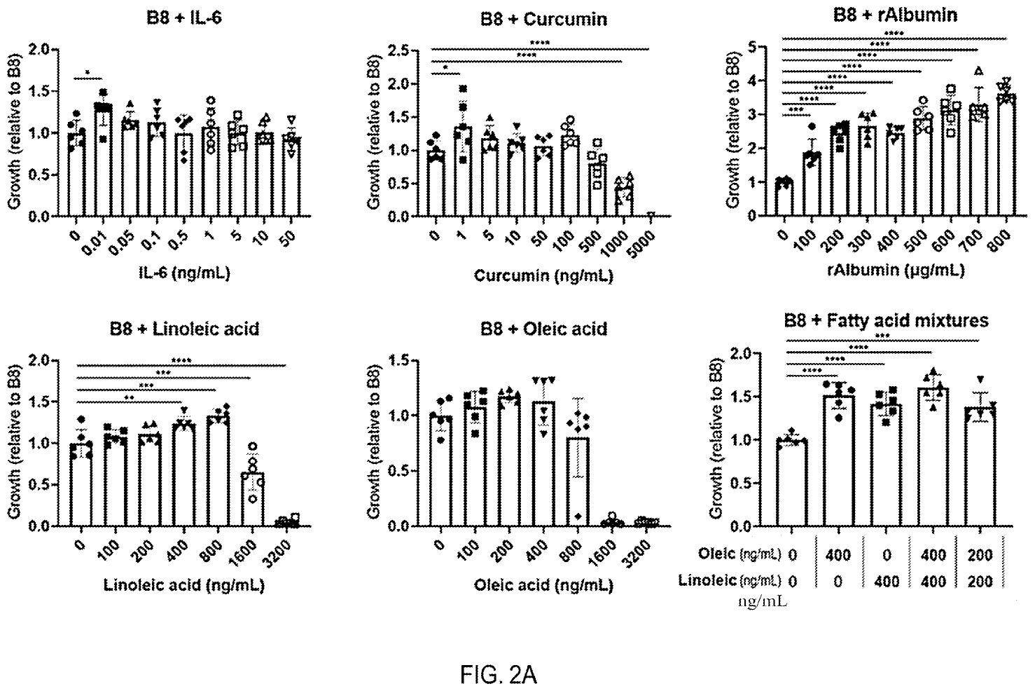

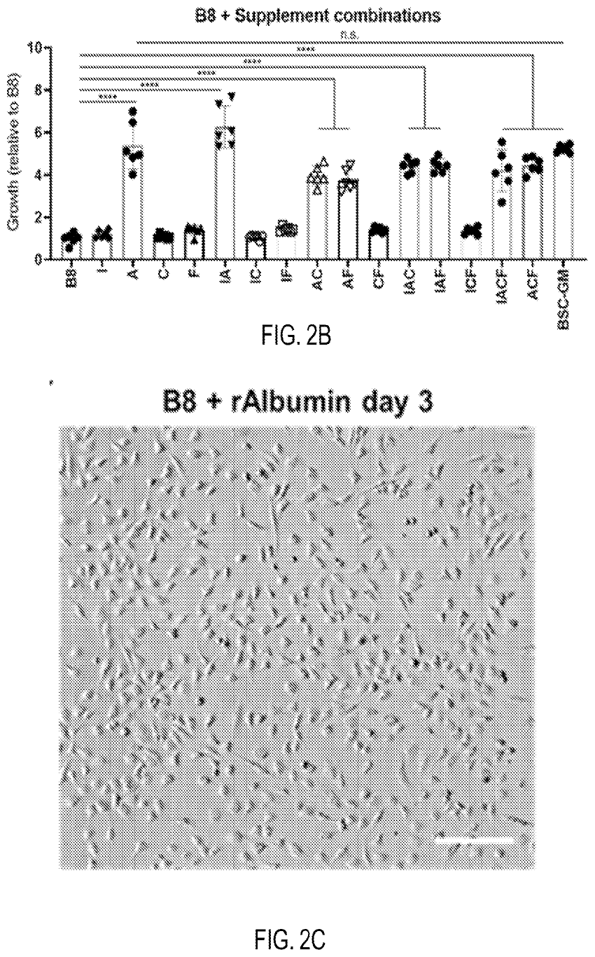

To overcome the deficiencies of B8 media alone, numerous supplements were tested in a range of concentrations (full list in Table 2), and growth was again analyzed over four days. Six of the factors significantly improved BSC proliferation compared to B8 media alone (FIG. 2A & data for those listed in Table 2 but absent from FIG. 2A can be provided if needed or can be located in the supplementary materials for a journal publication corresponding to this patent application). These were interleukin-6 (IL-6), curcumin, recombinant human albumin (rAlbumin), Platelet-derived growth factor (PDGF-BB), linoleic acid, and oleic acid. Of these, rAlbumin was particularly effective, imparting a ˜4-fold improvement in growth compared with plain B8. In contrast, other supplements resulted in at best only a ˜50% improvement compared with plain B8. To test whether combinations of these supplements could offer synergistic benefits to cell growth, optimal concentrations of the above factors (with the exception of PDGF-BB, which was determined to be insufficiently effective for the substantial cost) were combined and tested (FIG. 2B). Here, rAlbumin (800 μg/mL) was the driving factor in all significant improvements. While a combination of IL-6 (0.01 ng/mL) and rAlbumin offered slightly improved growth compared with rAbumin alone, this difference was not statistically significant. In the interest of maximizing media simplicity and minimizing cost, an augmented B8 media with nine components was established by supplementing with 800 μg/mL rAlbumin alone. This media was termed Beefy-9 due to its component number and design towards bovine muscle cell culture. This media was capable of maintaining short-term growth comparable to serum-containing BSC-GM and maintaining cell morphology in vitro (FIGS. 2B & C).

Passaging in Beefy-9 Media.

While short-term growth experiments were useful in establishing the benefits of rAlbumin supplementation for tailoring B8 media to BSC short term culture needs, long-term culture and passaging are essential for robust cell expansion required for cultured meat. However, seeding cells into Beefy-9 media directly after passaging initially proved ineffective, as cells did not re-adhere to tissue culture plastic. Two possible explanations were hypothesized for this result. The first was that the coating used (0.25 μg/cm² of laminin-511) was insufficient to enable cell adhesion in the absence of other adhesion factors present in serum (e.g., vitronectin and fibronectin). The second was that the high concentration of albumin was outcompeting laminin for adsorption to the tissue-culture plastic, thereby further hindering cell adhesion. To overcome these possible limitations, BSC passaging was explored in the absence of albumin and adding albumin one day after plating, in order to allow cells to adhere to the flasks, as well as passaging cells with various concentrations of different adhesive proteins. Because one priority of this work was media and cell culture workflow simplicity, focus was placed on recombinant adhesive proteins that were relatively low-cost and which have been demonstrated without pre-coating [e.g., laminin-511 fragment (iMatrix-511) and truncated vitronectin (Vtn-N)]. Poly-D-Lysine (PDL) coatings (which can be purchased commercially on pre-coated plates) were also explored to augment cell attachment with or without adhesive peptides. The results indicated that delaying the addition of rAlbumin was necessary to allow cell adhesion and growth, as is coating with a cell adhesive peptide such as iMatrix-511 (FIG. 3A). However, when comparing various cell adhesive peptides, the results indicated that iMatrix-511 was sub-optimal compared with Vtn-N (FIG. 3C). Specifically, 1.5 μg/cm² of Vtn-N showed superior cell adhesion (day 1) and growth (day 4) than PDL alone, laminin alone, PDL+laminin, or a lower concentration of Vtn-N with or without PDL. Once a suitable passaging method was determined, short-term growth curves were again performed with Beefy-9 supplemented with various growth factors in order to rule out the possible confounding effect that adsorbed serum proteins might have had on short term growth curves performed previously (in which cells were seeded in the presence of serum). No significant effect of these factors was found, reaffirming that supplementation with rAlbumin alone was optimal over multiple passages.

Serum-Free Differentiation Following Expansion in Beefy-9 Media.

After establishing delayed rAlbumin and 1.5 μg/cm² of Vtn-N as suitable parameters for multiple-passage culture of BSCs in Beefy-9, the myogenicity of expanded cells was determined. Specifically, Beefy-9-passaged cells (P2) were expanded to confluency in Beefy-9 and differentiated for 5 days in a previously published serum-free differentiation medium. See, Mcaleer, C. W., Rumsey, J. W., Stancescu, M. & Hickman, J. J. Functional myotube formation from adult rat satellite cells in a defined serum-free system. Biotechnol. Prog. 31, 997-1003 (2015). Differentiated cells showed the formation of multinucleated myotubes which stained positive for the myogenic marker myosin heavy chain (MHC) (FIG. 3C). The results validated that myogenicity of cells cultured and passaged in Beefy-9 media was maintained. Together, these results demonstrate a fully animal-component-free culture system for proliferating, passaging, and differentiating BSCs (video can be provided to a patent office, if needed, or the reader can locate a video in the supplementary material accompanying a journal article describing these experiments).

Cost-Reducing Strategies for Beefy-9 Media.

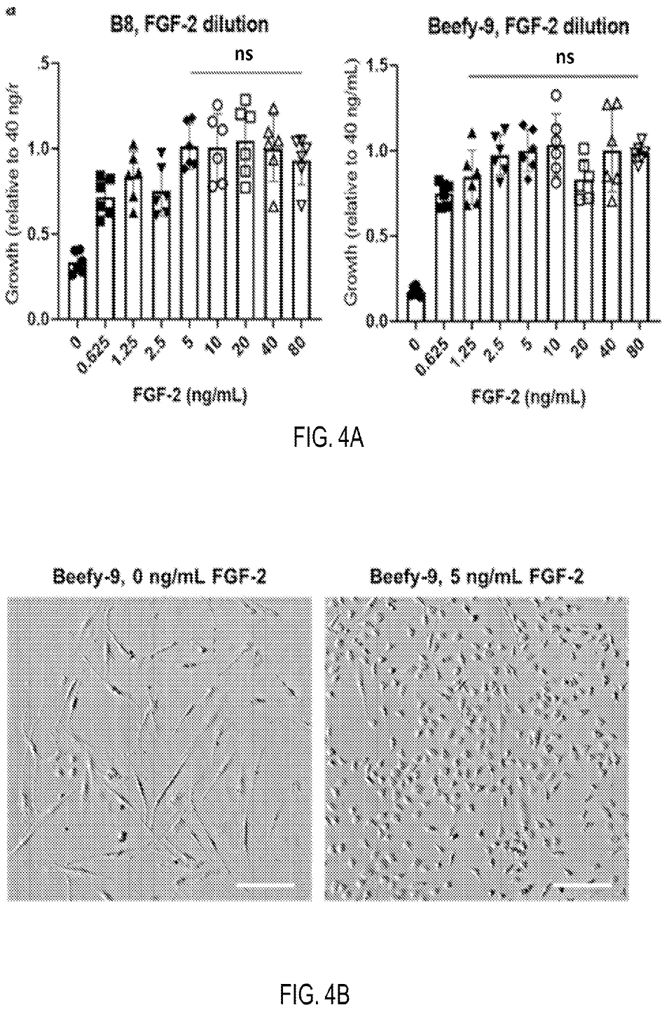

We next explored cost reduction strategies for Beefy-9 by lowering the concentration of FGF-2, which is a major cost contributor to B8 and Beefy-9 formulations at the baseline concentration of 40 ng/mL. Growth was analyzed over four days as before in B8 and Beefy-9 media with FGF-2 concentrations ranging from 0-80 ng/mL. The results showed that for B8 and Beefy-9, FGF-2 could be lowered to 5 ng/mL and 1.25 ng/mL, respectively, without significantly affecting growth (FIG. 4A). In contrast, cell growth and morphology were significantly affected by the complete removal of FGF-2 from the media (FIGS. 4A & B). The results indicated that substantial reduction in FGF-2 is possible to lower the cost of Beefy-9 without negatively impacting short-term growth rates.

Long-Term Culture in Beefy-9 Media.

The last step after validating Beefy-9 for short-term growth and establishing an appropriate passaging protocol was to validate long-term expansion of BSCs in Beefy-9. BSCs were seeded as before and fed with either serum-containing BSC-GM, B8, Beefy-9 with high FGF-2 (40 ng/mL), or Beefy-9 with low FGF-2 (5 ng/mL). The low FGF-2 concentration was conservatively selected as the concentration that did not significantly affect short-term growth in either B8 or Beefy-9. Cells were cultured and passaged as described (FIG. 3B) for seven passages over 28 days, with cell counts used to determine cumulative cell doublings over the four-week period. While BSC-GM was still the optimal media over a long growth period, Beefy-9 with high or low FGF-2 content showed significant improvements over B8 media without the addition of rAlbumin (FIG. 5A). Indeed, BSCs in B8 alone ceased proliferating after three passages (4.4 doublings), while BSCs in Beefy-9 continued to expand exponentially over at least seven passages (18.2 and 17.2 doublings for 40 ng/mL FGF-2 and 5 ng/mL FGF-2, respectively).

Converting growth data to doubling times revealed a consistent increase in doubling time over seven passages in Beefy-9 media, with a slower increase in doubling time (i.e., better-sustained proliferation) in BSC-GM (FIG. 5B); however, doubling times remained below 48 hours for Beef-9 media for the first five passages (13.7 doublings) and under 56 hours for all seven passages (18.2 doublings). The average doubling time over seven passages in Beefy-9 with high or low FGF-2 concentrations were ˜39 and ˜41 hours, respectively. These are higher than the doubling time of ˜17 hours that has been reported for satellite cells in vivo, but within a range expected based on previous reports of BSCs cultured in vitro. Together, these results indicated that the Beefy-9 media with both high and low FGF-2 concentrations were effective for the long-term expansion of bovine satellite cells, but that further optimization is needed to improve growth rates over multiple passages.

BSC Phenotype and Myogenicity Over Long-Term Culture.

Throughout long-term culture, the myogenicity of BSCs was verified in serum-containing and serum-free conditions. Cells from passages 2, 4, and 6 were cultured to confluency, differentiated in serum-free differentiation medium, and stained for Myosin Heavy Chain (MHC) as before. The formation of MHC-positive multinucleated myotubes in BSC-GM and Beefy-9 formulations over six passages was shown (images not shown, but can be provided to a patent office if needed). Interestingly, differentiation appeared improved in BSCs cultured in Beefy-9 media compared with BSC-GM over multiple passages, both in terms of myotube size and density, and in terms of quantitative fusion index (FIG. 8). This result could be because cells cultured in BSC-GM have undergone more doublings than cells cultured in Beefy-9 at the points of analysis, or because non-myogenic cells (e.g., fibroblast-like cells) overgrow myogenic satellite cells more rapidly in serum-containing media. While myogenicity was maintained throughout expansion in Beefy-9 media, it was noted that myotube diameter and density appears reduced at later passages. It was also noted that cells cultured in serum-free conditions appeared to accumulate lipid droplets over long-term culture, while cells cultured in BSC-GM did not (images not shown, but can be provided to a patent office if necessary). This aberrant lipid accumulation could be due to insulin resistance in cells as a result of the relatively high concentration of insulin in B8 & Beefy-9, and could therefore point towards a possible media optimization strategy by adjusting insulin levels. Alternately, lipid accumulation could suggest that BSCs are thrust towards an adipogenic phenotype in Beefy-9 media, though the sustained myogenicity of BSCs in serum-free conditions affirms the capacity of these media to maintain relevant satellite cell function for cultured meat. Ultimately, further exploration of this phenomenon is warranted in future studies.

Media Cost Analysis.

Once the efficacy of Beefy-9 was demonstrated, a simple cost analysis was performed to understand how this media might be implemented by research groups currently relying on serum-containing media (e.g., 20% FBS+1 ng/mL FGF-2, as used in this study) for cultured meat research. Price comparisons revealed that even using purchased growth factors and without bulk ordering (as in this study), Beefy-9 media cost substantially less than serum-containing media ($217/L vs. $290/L, respectively). At the reduced 5 ng/mL of FGF-2, the price of Beefy-9 drops further to $189/L). Further fold price decreases could easily be achieved by increasing the scale of culture media component orders, and by using powdered basal media. Specifically, the costs for Beefy-9 with high or low FGF-2 concentrations dropped to $74/L and $46/L, respectively, when components are ordered in bulk (full accounting of component sourcing can be provided to a patent examiner at their request or a reader can locate the supplementary materials of the journal article associated with the experiments described in this example). This amounts to a 75% cost reduction compared with bulk-ordered BSC-GM. In this study, Beefy-9 prices were dominated by rAlbumin, basal media, FGF-2 (at high concentrations), and insulin. If bulk ordering of store-bought components is used, the price was dominated by FGF-2 (at high concentrations), rAlbumin, and insulin, with basal media offering significantly less impact. While Beefy-9 is easy to produce in-house, further ease-of-use can be achieved by purchasing HiDef-B8 media and simply adding rAlbumin to prepare HiDef-Beefy-9; however, at current prices this results in a significant increase in cost.

Increasing rAlbumin Concentratin Improves Growth.

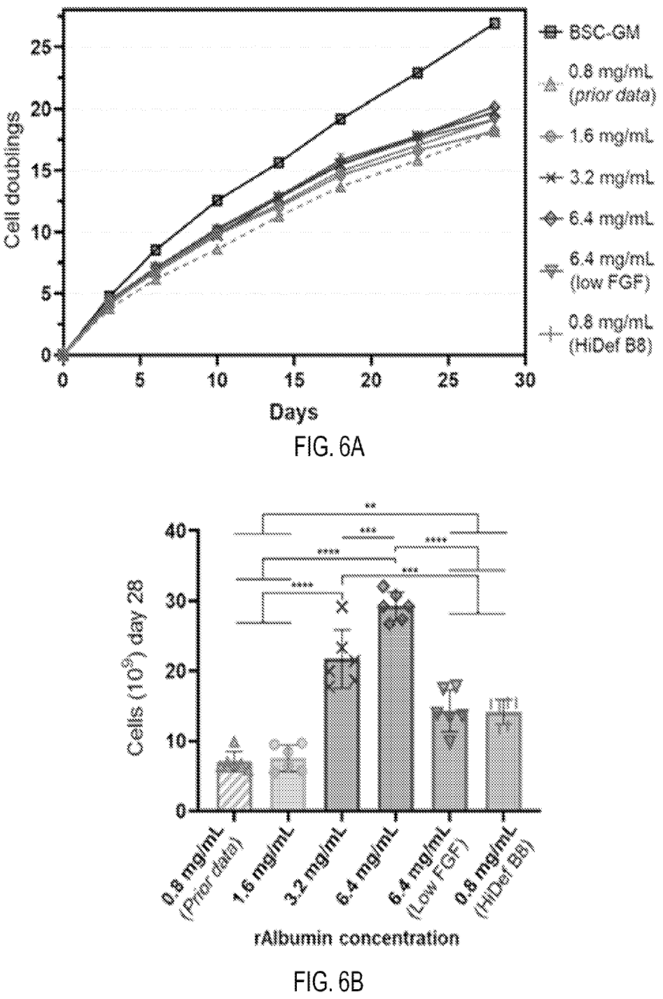

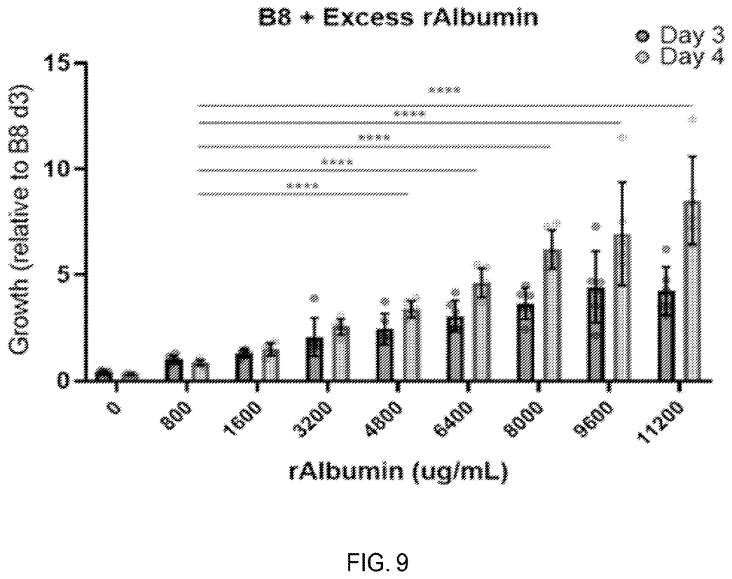

While 800 μg/mL of rAlbumin was used in the above work, short term growth analysis did not suggest that this was the optimal concentration of rAlbumin, merely the best of those initially tested. As increased rAlbumin resulted in improved growth (FIG. 2), it was investigated whether additional increases in rAlbumin could further improve outcomes. Short term analyses with concentrations of rAlbumin up to 11.2 mg/mL revealed continued improvements with increased rAlbumin, with the highest concentration yielding an 8.5-fold improvement in 4-day growth compared to Beefy-9's 800 ug/mL (0.8 mg/mL) (FIG. 9). In light of this, long-term growth was again investigated with increasing concentrtions of rAlbumin, and compared with the original Beefy-9 tests, HiDef Beefy-9 (using supplier-provided B8 with engineered growth factors), and BSC-GM (FIGS. 7A-B). While short-term growth analyses revealed that 11.2 mg/mL of rAlbumin offered the best results of the concentrations tested, cost analysis revealed that by 6.4 mg/mL rAlbumin, Beefy-9 had surpassed the cost of serum-containing BSC-GM. Because cost was a key factor in this study, long term growth analysis was therefore performed for rAlbumin concentrations up to, but not exceeding 6.4 mg/mL (FIG. 6A). Results showed that increased albumin improved growth over one month of cell expansion, with total doublings exceeding 19 and 20 for Beefy-9 containing 3.2 and 6.4 mg/mL rAlbumin, respectivley (compared with 18 for the original Beefy-9 containing 0.8 mg/mL rAlbumin). Taken as total cell count at day 28, these represent 3- and 4-fold improvements in cell yield, respectively (FIG. 6B). No improvement was observed for 1.6 mg/mL rAlbumin compared with 0.8 mg/mL at day 28, though a consistent improvement was seen for earlier passages. When comparing growth improvements with costs, increasing rAlbumin to 3.2 mg/mL resulted in a 3-fold improvement in cell number with only a 2-fold increase in cost. For 6.4 mg/mL rAlbumin, these values are 4-fold and 3-fold, respectively. As such, Beefy-9 with 3.2 mg/mL of rAlbumin (ter “Beefy-9+”) may offer the best ratio of cell growth to media cost (148 $/L), though the exact media used in a given scenario will likely depend on the specific constraints and priorities of the application in question. Throughout culture in Beefy-9 with additional rAlbumin, cells maintained their myogenicity, though the degree of MF20 staining as well as myotube density and diameter appear to have been reduced for cells grown in media containing 6.4 mg/mL rAlbumin (especially with reduced FGF). While Beefy-9+ with reduced FGF was not explored in this work, it is possible that this would further improve the performance/cost ratio. Additionally, the use of engineered growth factors (as in the original B8 work, and in supplier-provided HiDef B8) may improve performance. This was evidenced in long-term growth studies, where a 2-fold improvement in cell number was seen between HiDef Beefy-9 compared with standard Beefy-9.

Discussion.

Since its emergence, cultured meat research and development has been stymied by a lack of suitable serum-free media for muscle stem cell expansion. This deficiency has led the field to rely on FBS for most research, thereby hindering the relevance of findings. This is particularly the case when looking forward to serum-free production processes. Developing serum-free media for relevant cell types (e.g., muscle and fat) and relevant species (e.g., bovine, porcine, galline, etc.) is therefore essential to accelerate research in the field. These media should at a minimum be affordable, comprised of food-safe components, reliable, and easy to use (e.g., not overly complex) in order to promote their adoption in widespread research efforts and towards scaled production processes. In this work, two media (Beefy-9 and Beefy-9+) are described as promising candidates for simple serum-free culture of bovine satellite cells. This includes validation of: 1) the efficacy of these media in promoting BSC proliferation, 2) the ability of these media to enable long-term expansion when used in combination with rAlbumin-free B8 and truncated vitronectin, 3) the maintenance of satellite cell myogenicity when expanded in Beefy-9 and Beefy-9+, and 4) significant cost savings compared to serum-containing media.

The significant impact of rAlbumin on Beefy-9's efficacy for BSCs is noteworthy, particularly considering that the addition of albumin did not show nearly as profound of an effect on iPSC growth when B8 was originally developed. Albumin has many roles in cell culture media, and indeed comprises about 60% of the total proteins in serum. For an in-depth summary of these roles, the reader is directed to several thorough reviews. Briefly, though, albumin acts in culture media to bind, carry, and stabilize compounds such as fatty acids, metal ions, signaling molecules, amino acids, and other factors. As such, it is a potent and multifaceted antioxidant which can sequester these species from redox or other degrading reactions, increasing the half-life, availability, and solubility of beneficial factors to cells while reducing the accumulation of harmful byproducts. It is possible that these regulating effects impart an advantage to Beefy-9 and Beefy-9+ over B8 alone, as the latter was not incapable of promoting cell division in BSCs over the short term, only of fostering robust and prolonged expansion. Interestingly, albumin has also been suggested as a potential protectant against cell stresses due to sparging bubbles in various bioreactors, and so Beefy-9 media could offer additional benefits in these scaled-up bioprocesses.

A major goal of this work was to develop a medium that is simple and low-cost, as these two factors can help to lower the barriers to entry for cultured meat research, as well as lay the groundwork for a more diffuse production system (with less consolidation amongst a few concentrated corporations). Cost analysis suggests that Beefy-9 and Beefy-9+ media should be easy to implement and affordable (particularly in the case of Beefy-9) for academic labs that are currently using serum-containing (e.g., 20% FBS) media for cultured meat research. It should be noted, however, that significant cost reduction of serum-free media is still needed to make cultured meat production economically viable from an industrial perspective. Here, reducing the cost of albumin, growth factors and basal media (e.g., through the use of plant or algal hydrolysates) will be essential. Additionally, co-culture of meat-relevant cells with nutrient- or growth factor-producing cells could offer valuable cost-saving opportunities. When considering Beefy-9, it is clear that recombinant proteins are the main drivers of cost. As such, further research should explore cost-reduction of these recombinant proteins, or opportunities for substituting or eliminating these factors. Additionally, it should be noted that in the absence of cell-adhesion proteins found in FBS, a significant increase in the concentration of tissue-vessel coating was required to achieve adequate BSC adhesion and growth during serum-free culture. This factor is often overlooked when discussing the cost of large-scale cell culture; however, the present study relied on 1.5 μg/cm² of Vtn-N, which adds ˜$0.18/cm², or $31.75 per T-175 flask (a standard vessel used in our lab, suitable for ˜5 doublings at standard seeding and passaging densities). Opportunities to reduce the costs associated with cell adhesion include adapting or engineering cells to suspension culture, pre-coating flasks with Vtn-N with or without additional factors, reducing the cost of recombinant adhesion protein production, or exploring low-cost alternatives to recombinant production.

While this work presents a promising resource for researchers and a useful foundation for further media development, it is clear that further media optimization for both cost and long-term efficacy is warranted. Specifically, the present work relies on one-factor-at-a-time exploration of media components to find suitable supplements to tailor B8 for bovine muscle stem cells. As media components are intimately entangled in their effects on cell biology, it is therefore unlikely that Beefy-9 or Beefy-9+ are optimal as-is. Indeed, it is possible that media components which appeared inconsequential in this work (e.g., hepatocyte growth factor or ethanolamine) would offer advantages in other permutations of media not tested in the development of Beefy-9. Computational approaches to media development are better suited for solving such multi-factorial problems, and should therefore be leveraged to further optimize Beefy-9 and other serum-free media for cultured meat applications. Here, efforts can focus on adipose as well as muscle tissue, for instance through the use of free fatty acid addition to Beefy-9 to induce the transdifferentiation of BSCs into lipid-accumulating cells. Additionally, further work is needed to overcome the slower long-term growth and aberrant lipid accumulation that is seen in BSCs cultured over seven passages in Beefy-9. Promising options for this include the use of spontaneously or genetically immortalized stem cells, which could improve long-term outcomes in Beefy-9, and the exploration of different concentrations of insulin, fatty acids, or other signaling factors to better control differentiation down myogenic vs. adipogenic pathways.

The present work offers a simple, affordable, and effective Beefy-9 and Beefy-9+ serum-free media for improving cultured meat research. However, the cost and efficacy of these media will require further optimization for industrial scale production of cultured meats that aim to reach price-parity with conventional meats. Future work to address these needs could focus on both engineering efforts (e.g., increased recombinant growth factor productivity, production of species-specific recombinant proteins, optimized media formulations, media recycling, and cell line engineering) and scientific discoveries (e.g., novel protein analogues or alternatives, or insights into cell signaling pathways) in order to drive the cost of production down. Additionally, exploration of cell sourcing and isolation techniques, cell lineage control (e.g., of myogenic vs. adipogenic differentiation), and bioreactor systems can build on substantial prior work and help to increase production efficiency and drive down costs. Here, cultured meat development is likely to provide collateral benefit to biomedical research, such as tissue engineering for volumetric muscle loss or cell-based biopharmaceutical production. Ultimately, sustained efforts towards serum-free media development are needed to continually lower costs and improve scalability of cultured meat over time, bringing products closer to market viability and cultured meat's possible benefits closer to reality.

1. A serum-free and animal-component-free culture media for expansion of muscle satellite cells for use in cultured food applications, the media comprising:
a baseline serum-free and animal-component-free culture media that, in use, provides a baseline growth capability for expansion of the muscle satellite cells for use in cultured food applications;
a recombinant version of albumin,
wherein the serum-free and animal-component-free culture media, in use, provides an improved growth capability for expansion of the muscle satellite cells for use in cultured food applications,
wherein the improved growth capability is at least 50% greater than the baseline growth capability.

2. The serum-free and animal-component-free culture media of claim 1, wherein the baseline serum-free and animal-component free culture media comprises:
basal media;
one or more sugars at a concentration of between 0.01 g/L and 10 g/L;
one or more amino acids at a concentration of between 0.001 g/L and 5 g/L;
one or more vitamins at a concentration of between 0.001 g/L and 1 g/L;
one or more optional minerals at a concentration of between 0 g/L and 15 g/L;
one or more trace elements at a concentration of between 0.0001 mg/L and 10 mg/L;
one or more growth-stimulating and/or cell-signaling factors at a concentration of between 0.01 ng/mL and 100,000 ng/mL; and
one or more carrying proteins at a concentration of between 0.01 g/L and 1 g/L.

3. The serum-free and animal-component-free culture media of claim 1, the serum-free and animal-component-free culture media further comprising a recombinant version of one or more of the following:
interleukin 6;
ethanolamine;
curcumin;
oleic acid; or
linoleic acid.

4. The serum-free and animal-component-free culture media of claim 1, wherein the recombinant version of albumin is present in an amount of at least 50 mg/L, at least 100 mg/L, at least 200 mg/L, at least 250 mg/L, at least 500 mg/L, at least 750 mg/L, at least 1 g/L, at least 2 g/L, at least 5 g/L, at least 10 g/L, at least 15 g/L, or at least 20 g/L and at most 60 g/L, at most 50 g/L, at most 40 g/L, at most 30 g/L, at most 25 g/L, at most 20 g/L, at most 15 g/L, at most 10 g/L, at most 5 g/L, at most 2 g/L, at most 1.5 g/L, or at most 1 g/L.

5. The serum-free and animal-component-free culture media of claim 1, wherein the recombinant version of albumin is present in an amount of about 800 mg/L.

6. The serum-free and animal-component-free culture media of claim 1, wherein the recombinant version of albumin is a recombinant version of human albumin.

7. The serum-free and animal-component-free culture media of claim 3, comprising the recombinant version of interleukin 6.

8. The serum-free and animal-component-free culture media of claim 3, comprising the recombinant version of ethanolamine.

9. The serum-free and animal-component-free culture media of claim 3, comprising the recombinant version of curcumin.

10. The serum-free and animal-component-free culture media of claim 3, comprising the recombinant version of oleic acid.

11. The serum-free and animal-component-free culture media of claim 3, comprising the recombinant version of linoleic acid.

12. The serum-free and animal-component-free culture media of claim 1, wherein the baseline serum-free and animal-component-free culture media is B8 media.

13. The serum-free and animal-component-free culture media of claim 1, comprising a basal media, L-ascorbic acid 2-phosphate, recombinant insulin, recombinant Transferrin, sodium selenite, recombinant fibroblast growth factor 2, recombinant Neregulin 1, and recombinant transforming growth factor beta 3.

14. The serum-free and animal-component-free culture media of claim 1, wherein the muscle satellite cells are bovine, galline, ovine, porcine, equine, murine, caprine, lapine, or piscine.

15. The serum-free and animal-component-free culture media of claim 1, wherein the muscle satellite cells are bovine, galline, porcine, or piscine.

16. A method of making a serum-free and animal-component-free culture media, the method comprising adding a recombinant albumin to a baseline serum-free and animal-component-free culture media, thereby producing the serum-free and animal-component-free culture media.

17. A method of making an engineered cell, the method comprising expanding muscle satellite cells in the serum-free and animal-component-free culture media of claim 1.

18. The method of claim 17, the method further comprising:
prior to expanding the muscle satellite cells, either: i) coating the muscle satellite cells with a recombinant version of a cell adhesive peptide; or ii) adhering the muscle satellite cells in the baseline serum-free and animal-component-free culture media and/or a different baseline serum-free and animal-component-free culture media that is lacking the recombinant version of albumin.

19. The method of claim 18, the method comprising the coating of step i) and the adhering of step ii).

20. The method of claim 18, wherein the recombinant version of the cell adhesive peptide is selected from the group consisting of a recombinant version of a laminin or fragments thereof, vitronectin or fragments thereof, poly-d-lysine, poly-l-lysine, fibronectin or fragments thereof, Matrigel, and combinations thereof.
